(19) United States
(12) Patent Application Publication
(10) Pub. No.: US 2009/0325256 A1
(43) Pub. Date: Dec. 31, 2009

(54) METHOD FOR CELL PATTERNING

(75) Inventors: Tomoyuki Yasukawa, Sendai-shi (JP); Masato Suzuki, Sendai-shi (JP); Hitoshi Shiku, Sendai-shi (JP); Yoshio Hori, Sendai-shi (JP); Akiko Inagaki, Sendai-shi (JP); Mariko Komabayashi, Sendai-shi (JP); Tomokazu Matsue, Sendai-shi (JP)

Correspondence Address: OLIFF & BERRIDGE, PLC, P.O. BOX 320850, ALEXANDRIA, VA, 22320-4850, US

(73) Assignee: TOHOKU UNIVERSITY, Sendai-shi (JP)

(21) Appl. No.: 12/310,042

(22) PCT Filed: Aug. 3, 2007
(86) PCT No.: PCT/JP2007/065294

§ 371 Date: Apr. 27, 2009

(30) Foreign Application Priority Data
Aug. 10, 2006 (JP) 2006-218280

(51) Int. Cl. C12N 13/00

(52) U.S. Cl. 435/173.1

(57) ABSTRACT

Provided is a method for cell patterning, using an electrode substrate including a plurality of electrodes and a cell culture substrate disposed so as to face the electrode substrate, the method comprising the steps of:
introducing a cell suspension containing cells into a region between the electrode substrate and the cell culture substrate; applying a voltage to the electrodes to generate a non-uniform electric field in the region; and arranging the cells at a position with low electric field strength on the cell culture substrate by utilizing negative dielectrophoresis so as to obtain the cell culture substrate on which the cells are arranged in a predetermined pattern.

### TECHNICAL FIELD

The present invention relates to a method for cell patterning.

### BACKGROUND OF THE INVENTION

It has been expected that techniques for performing in vitro reconstruction of an in vivo cellular environment are applied to various fields such as cell biological analysis of cell function, personalized drug screening by use of a cell array chip, elucidation of intercellular communication and cell-extracellular matrix communication for regenerative medicine. A technique for performing in vitro reconstruction of an in vivo cellular environment which has attracted attention is a cell patterning technique which is a technique for disposing cells, extracellular matrices, and cell adhesion molecules in any region at a micro-scale.

For example, Japanese Unexamined Patent Application Publication No. Hei 2-245181 (Document 1) discloses a method for cell patterning utilizing an electrostatic charge pattern. In this method for cell patterning, a body tissue is attached onto a charge retaining medium on which an electrostatic charge pattern is formed, and cells are cultured while utilizing ionic interaction of the tissue. In addition, Japanese Unexamined Patent Application Publication No. Hei 5-176753 (Document 2) discloses a cell culture substrate usable in a method for cell patterning. This cell culture substrate has a surface part onto which a substance specifically influential on cell adhesion rate and cell adhesion morphology adsorbs. Moreover, Japanese Unexamined Patent Application Publication No. 2005-143382 (Document 3) discloses a cell culture substrate including a base member and a cell culture patterning layer formed on the base member. The cell culture patterning layer includes at least a photocatalyst and a cell adhesion material that has adhesive properties to cells and that is decomposed or modified by action of the photocatalyst upon energy irradiation. Furthermore, as a method for performing cell separation or the like, Japanese Unexamined Patent Application Publication No. 2004-522452 (Document 4) discloses a method for separating cells by dielectrophoresis. Further, Japanese Unexamined Patent Application Publication No. 2005-249407 (Document 5) discloses a hybridization method in which a biopolymer is concentrated in the vicinity of a conduction path by dielectrophoresis.

### DISCLOSURE OF THE INVENTION

In the method for cell patterning described in Document 1, however, a process for producing the cell culture substrate is complicated, and cells are not efficiently patterned. In addition, with the method as described in Document 1, it is difficult to pattern plural kinds of cells onto one substrate. As for the cell culture substrates as described in Documents 2 and 3, the production processes thereof are complicated, because it is necessary to form a micrometer-order pattern on these substrates in production of these substrates. Moreover, when such a substrate as described in Document 2 or 3 is used, it is also difficult to pattern plural kinds of cells onto one substrate. Further, when cells are patterned by using a method as described in Document 4 or 5, the cell culture substrate is also used as an electrode substrate that induces the dielectrophoresis phenomenon, and thus cells are arranged onto the electrode substrate which has been produced through the complicated processes. Accordingly, it is difficult to reuse the electrode substrate. In addition, when cells are patterned by using the method as described Document 4 or 5, it is difficult to pattern plural kinds of cells onto one substrate.

The present invention has been made in consideration of the above-described problems in the conventional techniques. An object of the present invention is to provide a method for cell patterning which: eliminates the need for forming, in advance, a pattern on a cell culture substrate in order to arrange cells; allows cells to be efficiently arranged onto the cell culture substrate in a predetermined pattern; and enables an electrode substrate to be used repeatedly by detaching the electrode substrate from the cell culture substrate.

The present inventors have earnestly studied in order to achieve the above object. As a result, the inventors have revealed that it is possible: to eliminate the need for forming, in advance, a pattern on a cell culture substrate in order to arrange cells; to efficiently arrange cells onto the cell culture substrate in a predetermined pattern; and to repeatedly use an electrode substrate by detaching the electrode substrate from the cell culture substrate. This is made possible in the following manner. Specifically, by using an electrode substrate including a plurality of electrodes and a cell culture substrate disposed so as to face the electrode substrate, a cell suspension containing cells is introduced into a region between the electrode substrate and the cell culture substrate; a voltage is applied to the electrodes to generate an non-uniform electric field in the region; and the cells are arranged at a position with low electric field strength on the cell culture substrate by utilizing negative dielectrophoresis. This discovery has led the inventors to complete the present invention.

Specifically, the method for cell patterning of the present invention is a method using an electrode substrate including a plurality of electrodes and a cell culture substrate disposed so as to face the electrode substrate, the method comprising the steps of: introducing a cell suspension containing cells into a region between the electrode substrate and the cell culture substrate; applying a voltage to the electrodes to generate a non-uniform electric field in the region; and arranging the cells at a position with low electric field strength on the cell culture substrate by utilizing negative dielectrophoresis so as to obtain the cell culture substrate on which the cells are arranged in a predetermined pattern.

In addition, in the method for cell patterning of the present invention, a plurality of cell suspensions are preferably prepared as the cell suspension, the plurality of cell suspensions are preferably introduced one after another into the region, and, by selecting a predetermined position with a large dielectrophoretic force depending on cells in each suspension, a plurality of cells are preferably arranged onto the cell culture substrate one after another so as to obtain the cell culture substrate on which the plurality of cells are arranged in a predetermined pattern.

In the method for cell patterning of the present invention, in a case where the plurality of electrodes generate a plurality of electric fields having electric-field-strength maximum values of 8×10⁴ V/m or more on the cell culture substrate, the position with low electric field strength is preferably a halfway region between maximum points of the electric fields being adjacent to each other, and satisfying conditions that an electric-field-strength maximum value of each electric field is 8×10⁴ V/m or more (more preferably, 8×10⁴ to 10×10⁴ V/m, especially preferably, approximately 9×10⁴ V/m), and that a space between the electric-field-strength maximum points is 30 to 200 μm (more preferably 30 to 150 μm).

In addition, in the method for cell patterning of the present invention, a distance between the electrode substrate and the cell culture substrate is preferably 30 to 50 μm.

Furthermore, in the method for cell patterning of the present invention, a content of the cell in the cell suspension is preferably 5×10⁷ cells/ml or less, and a solvent for the cell suspension preferably has a polarizability larger than a polarizability of the cells.

Here, it is not known exactly why the method for cell patterning of the present invention can achieve the above object. However, the present inventors speculate as follows. Specifically, in the present invention, a cell suspension containing cells is first introduced into the region between the electrode substrate and the cell culture substrate, and an alternating voltage is applied to the region to generate a non-uniform electric field therein. Such application of the voltage induces a dipole moment that is attributable to the difference in polarizability between cells and a solvent. Next, the interaction between the induced dipole moment and a difference in electric field strength results in repulsive forces acting on the cells. Among phenomena in which such repulsive forces act, a phenomenon of negative dielectrophoresis is used to arrange cells onto the cell culture substrate in the present invention. In the negative dielectrophoresis, cells in a region with high electric field strength are guided to a region with low electric field strength when subjected to repulsive forces. Thus, in the present invention, it is possible to arrange cells at a position with low electric field strength in a predetermined pattern without conducting a special pre-treatment on the cell culture substrate. Further, in the present invention, cells are guided to the region with low electric field strength and arranged therein. Thus, the pattern into which the cells are arranged can be changed easily, by controlling the combination of electrodes to which a voltage is applied and appropriately changing the position with low electric field strength. Moreover, when multiple cell suspensions are prepared and introduced one after another and then the position with low electric field strength is changed appropriately to arrange the cells, it is further possible to arrange easily and separately plural kinds of cells at any positions, which allows an easy patterned co-culture of the plural kinds of cells. Furthermore, in the present invention, the cell culture substrate on which cells are to be arranged and the electrode substrate are separated, and the cells are arranged onto the cell culture substrate. Thus, the electrode substrate can be used repeatedly.

The present invention can provide a method for cell patterning which; eliminates the need for forming, in advance, a pattern on a cell culture substrate in order to arrange cells; allows cells to be efficiently arranged onto the cell culture substrate in a predetermined pattern; and enables an electrode substrate to be used repeatedly by detaching the electrode substrate from the cell culture substrate. Further, the present invention makes it possible to arrange plural kinds of cells in a predetermined pattern, which allows a patterned co-culture of the plural kinds of cells.

### BRIEF DESCRIPTION OF THE DRAWINGS

FIG. 1 a schematic view showing a preferred embodiment of an apparatus that can be used in a method for cell patterning of the present invention.

FIG. 2 is a schematic view showing the preferred embodiment of the apparatus shown in FIG. 1 in a case where the method for cell patterning of the present invention is implemented.

FIG. 3 is a schematic view showing the preferred embodiment of the apparatus shown in FIG. 1 in a case where the method for cell patterning of the present invention is implemented.

FIG. 4 is a schematic view showing a preferred embodiment of a cell culture substrate obtained when cells are patterned by use of the apparatus shown in FIG. 1.

FIG. 5 is an outline view of a process for producing an electrode substrate that can be suitably used in the present invention. FIG. 5(a) shows an outline view of an ITO electrode substrate. FIG. 5(b) shows an outline view of the ITO electrode substrate on which an IDA pattern (electrode wirings) is formed. FIG. 5(c) shows an outline view of the ITO electrode substrate on which a bridge straddling the electrode wirings is formed. FIG. 5(d) shows an outline view of the ITO electrode substrate on which a gold electrode that straddles the bridge and the underlying electrode wirings.

FIG. 6 is a drawing showing an optical micrograph (FIG. 6(a)) of an IDA electrode (electrode substrate) of four-independently-operating-electrode type produced in Production Example 1, and a cyclic voltammogram (FIG. 6(b)) of the electrode substrate.

FIG. 7 is a graph showing analysis results of electric field strength conducted in a case where an IDA electrode of four-independently-operating-electrode type is used in a cell patterning apparatus produced in production Example 2 having a model with dimensions of length (x axis) 900 μm×width (y axis) 10 μm×height (z axis) 30 μm. FIG. 7(a) is a graph showing the electrode strength of a section represented in a gray scale in a case where an electrode (ii) in the electrode substrate is used as the positive electrode and electrodes (i), (iii) and (iv) are used as the negative electrodes. FIG. 7(b) is a graph showing the relationship between the x axis and the electric field strength in a plane located at a height (the z axis) of 30 μm from the electrode substrate in a case were the electrode (ii) in the electrode substrate is used as the positive electrode and the electrodes (i), (iii), and (iv) are used as the negative electrodes. FIG. 7(c) is a graph showing the electrode strength of a section represented in a gray scale in a case where an electrode (iv) in the electrode substrate is used as the positive electrode and electrodes (i), (ii) and (iii) are used as the negative electrodes. FIG. 7(d) is a graph showing the relationship between the x axis and the electric field strength in a plane located at a height of 30 μm from the electrode substrate in a case where the electrode (iv) in the electrode substrate is used as the positive electrode and the electrodes (i), (ii) and (iii) are used as the negative electrodes.

FIG. 8(a) is an optical micrograph of a cell culture substrate onto which polystyrene fine particles were patterned by negative dielectrophoresis by use of the cell patterning apparatus produced in Production Example 2, in which the electrode (ii) in the electrode substrate was used as the positive electrode and the electrodes (i), (iii) and (iv) were used as the negative electrodes. FIG. 8(b) is an optical micrograph of a cell culture substrate onto which polystyrene fine particles were patterned by negative dielectrophoresis by use of the cell patterning apparatus produced in Production Example 2, in which the electrode (iv) in the electrode substrate was used as the positive electrode and the electrodes (i), (ii) and (iii) were used as the negative electrodes.

FIG. 9 is a graph (FIG. 9(a)) showing the relationship between the electrical conductivity of a medium and a frequency (a cross-over frequency), and a graph (FIG. 9(b)) showing the relationship between a frequency and Re([K].

FIG. 10(a) is an optical micrograph of a cell culture substrate obtained by being detached from the apparatus after arrangement of cells in Example 1, this optical micrograph being taken immediately after the detachment. FIG. 10(b) is an optical micrograph showing the cell culture substrate at the time when the cells were cultured by immersing the cell culture substrate shown in FIG. 10(a) into a medium, this optical micrograph being taken one hour after start of the culture. FIG. 10(c) is an optical micrograph showing the cell culture substrate at the time when the cells were cultured by immersing the cell culture substrate shown in FIG. 10(a) into the medium, this optical micrograph being taken 22 hours after the start of the culture. FIG. 10(d) is an optical micrograph showing the cell culture substrate at the time when the cells were cultured by immersing the cell culture substrate shown in FIG. 10(a) into the medium, this optical micrograph being taken 9 days after the start of the culture.

FIG. 11 is a graph showing the relationship between a voltage and pattern efficiency (eₚ) in a case where the cell patterning apparatus (Production Example 2) used in is Example 1 was used.

FIG. 12(a) is an optical micrograph of a cell culture substrate obtained in Example 3. FIG. 12(b) is an optical micrograph showing a state where cells on the cell culture substrate obtained in Example 3 were caused to emit fluorescence. FIG. 12(c) is an optical micrograph of a cell culture substrate obtained in Example 4. FIG. 12(d) is an optical micrograph showing a state where cells on the cell culture substrate obtained in Example 4 were caused to emit fluorescence.

### DETAILED DESCRIPTION OF THE PREFERRED EMBODIMENTS

Hereinafter, preferred embodiments of the present invention will be described in details with reference to the drawings. Note that, in the following description and drawings, the same or corresponding elements are denoted by the same reference symbols, and redundant description will be omitted.

A method for cell patterning of the present invention is a method using an electrode substrate including a plurality of electrodes and a cell culture substrate disposed so as to face the electrode substrate, the method comprising the steps of: introducing a cell suspension containing cells into a region between the electrode substrate and the cell culture substrate; applying a voltage to the electrodes to generate a non-uniform electric field in the region; and arranging the cells at a position with low electric field strength on the cell culture substrate by utilizing negative dielectrophoresis so as to obtain the cell culture substrate on which the cells are arranged in a predetermined pattern.

First, description will be given of a preferred embodiment of an apparatus that can be used in the implementation of the method for cell patterning of the present invention. FIG. 1 is a schematic view showing the preferred embodiment of the apparatus that can be used in the method for cell patterning of the present invention. The apparatus shown in FIG. 1 includes: an electrode substrate 1 provided with plural electrodes 2; a cell culture substrate 3; and a spacer 4. The cell culture substrate 3 is disposed so as to face the electrode substrate 1 with the spacer 4 interposed therebetween.

Such an electrode substrate 1 includes the multiple electrodes 2 formed thereon. When a voltage is applied to the electrodes 2, the electrode substrate 1 allows a non-uniform electric field to be generated in a region between the electrode substrate 1 and the cell culture substrate 3. Such an electrode substrate 1 is not particularly limited, and the design thereof can be modified as appropriate in accordance with a cell pattern to be formed. Further, a method for producing such an electrode substrate is not particularly limited, and the electrode substrate can be produced as appropriate by a publicly-known method. For example, the electrode substrate may be produced by forming electrodes on a substrate by use of a photoresist or the like. A material for the electrode substrate 1 is not particularly limited, as long as the material allows the electrodes to be wired on the substrate. Accordingly, publicly-known materials can be used as appropriate for the electrode substrate 1. Further, the design of the electrodes formed on the electrode substrate 1 is not particularly limited, as long as the design allows generation of a region with a low electric field on the cell culture substrate 3. The design can be modified as appropriate in accordance with a cell pattern to be formed.

Moreover, the cell culture substrate 3 is not particularly limited, as long as the cell culture substrate 3 allows cells to be cultured thereon. Publicly-known cell culture substrates can be used as the cell culture substrate 3 as appropriate. For example, a plastic Petri dish for cell culture can be suitably used. A conventionally-used substrate on which a micrometer-order pattern to arrange cells is formed in advance by use of a photoresist or the like does not need to be used as the cell culture substrate 3, and a publicly-known cell culture substrate can be used as it is. In this way, the present invention eliminates the need for pre-treating the cell culture substrate with a photoresist or the like. Thus, cells can be arranged efficiently.

Furthermore, it is only necessary for the spacer 4 to be capable of forming a space that allows a cell suspension to be introduced into the region between the electrode substrate 1 and the cell culture substrate 3. The shape, material, and the like of the spacer 4 are not particularly limited, and the design of the spacer 4 can be modified as appropriate for use in accordance with the shapes or the like of the electrode substrate 1 or the cell culture substrate 3.

In addition, the distance between the electrode substrate 1 and the cell culture substrate 3 is not particularly limited, since the optimum distance varies depending on; the kinds of cells and a solvent to be used; the design of the apparatus; the magnitude and the frequency of an alternating voltage to be applied; and the like. The distance, however, is preferably approximately 30 to 50 μm. If the distance falls bellow 30 μm, a pattering precision tends to be impaired due to frequent nonspecific adhesion of cells onto the electrode substrate 1 or the cell culture substrate 3. This is because the cells are frequently brought into contact with the electrode substrate 1 or the cell culture substrate 3. On the other hand, if the distance exceeds 50 μm, patterned cells tend not to adhere to the cell culture substrate 3, and further a cell pattern tends to blur. These are due to emergence of low dielectrophoretic force in a wide region.

Next, as a preferred embodiment of the method for cell patterning of the present invention, description will be made of a method for cell patterning in which the above-described apparatus shown in FIG. 1 is used.

In such a method for cell patterning, first, a cell suspension containing cells is introduced into the region between the electrode substrate 1 and the cell culture substrate 3.

Such a cell suspension is not particularly limited, as long as the cell suspension contains cells to be patterned. A cell suspension prepared by a publicly-known method can be used as appropriate. In addition, a solvent for such a cell suspension is not particularly limited, and a solvent selected from publicly-known solvents can be used as appropriate in accordance with cells to be used. Moreover, it is preferable that a solvent with a polarizability greater than the polarizability of the cells be used as the solvent, since the method utilizes negative dielectrophoresis. The cell content in the cell suspension is not particularly limited; however, the cell content is preferably 5×10⁷ cells/ml or less. If the cell content exceeds the upper limit, it tends to be difficult to form a target pattern. This is because, although part of the cells is integrated with each other in a region with low electric field strength, the rest of the cells are present in regions other than the region with low electric field strength.

Moreover, a method for introducing such a cell suspension is not particularly limited, as long as the method allows the cell suspension to be introduced into the region between the electrode substrate 1 and the cell culture substrate 3. The method may be a batch method or a flow method.

Next, after the cell suspension containing cells is introduced into the region, a voltage is applied to the electrodes 2. Accordingly, a non-uniform electric field is generated in the region, and the cells are arranged at a position with low electric field strength on the cell culture substrate 3 by utilizing negative a dielectrophoresis. Thus, the cell culture substrate 3 on which the cells are arranged in a predetermined pattern is obtained.

The strength, frequency, and the like of the voltage applied as described above are not particularly limited.

The optimum values of the strength, frequency, and the like can be set as appropriate in accordance with; the distance between the electrode substrate 1 and the cell culture substrate 3; the design of the apparatus, for example the shape of the electrode substrate 1; the design of the cell suspension, for example the kind of cells and the kind of the solvent; and the like. Note that, when a large voltage is applied, damage on the cells tends to be caused by an electric field, although adhesion of many cells onto the culture substrate is promoted. Note also that, when a small voltage is applied, pattern tends not to remain on the cell culture substrate, although the electrical damage on the cells is reduced. Thus, in order to optimize the voltage to be applied, the following method may be adopted, for example. Specifically, in the method, voltages with different strength are applied independently, and the cell pattern formation ratios eₚ at the respective voltages are measured in advance. Thereafter, voltage strength suitable for arranging the cells is derived on the basis of the data. Here, the pattern formation ratio eₚ is the value represented by the following formula (1):

eₚ=n₁ₕᵣ/nₜₒₜₐₗ (1)

(where, nₜₒₜₐₗ represents the number of the cells existing on micro-band electrodes after termination of a 5 minute-voltage application, and n₁ₕᵣ represents the number of the cells existing on a culture slide after the cells are cultured for one hour). Note that cell number determination methods adaptable herein are: a determination method in which cells present on a culture slide are subjected to fluorescence staining thereby enabling the cells to be observed and counted; and a determination method in which cells on the culture slide are subjected to microscopic observation and then counted. Accordingly, n₁ₕᵣ in the above formula (1) represents either the number of cells present on the culture slide observed after the cells are cultured for one hour and subjected to fluorescence staining, or the number of cells obtained by counting cells present on the culture slide with microscopic observation of the cells after the cells are cultured for one hour.

In the present invention, cells are guided to a predetermined position by negative dielectrophoresis that is enabled by applying a voltage to the electrodes 2 to generate a non-uniform electric field. In the present invention, since negative dielectrophoresis is utilized as described above, no cell pattering is performed on the electrode substrate. Accordingly, the electrode substrate can be used repeatedly, and thus the cells can be more efficiently patterned.

Here, brief description will be made of dielectrophoresis. Dielectrophoresis is a phenomenon in which a force acts on a cell as a result of the interaction between a non-uniform electric field applied from the outside and the dipole moments of the cell and a solvent induced by that electric field (Pohl, Jones, Morgan, and Hughes). Accordingly, the direction of the force acting on the cell varies in accordance with the state of the cell surface. For example, a case where the cell is guided to a region with high electric field strength is referred to as positive dielectrophoresis. On the other hand, a case where the cell is guided to a region with low electric field strength is referred to as negative dielectrophoresis. The frequency of the voltage applied form the outside, the electrical conductivity of the solution, the surface charge state of the cell, and the like determine which of the positive dielectrophoresis and the negative dielectrophoresis actually occur. In addition, the dielectrophoretic force in such dielectrophoresis is defined by the following formula (2):

[Expression 1]

[symbol]

F̅_{DEP} [symbol] =2π∈ₛr³Re[K(ω)]∇E²ᵣₘₛ (2)

(where, r represents the radius of a particle, ∈_{c} represents the dielectric constant of a solvent in a suspension, the nabla symbol represents a vector operator, Eᵣₘₛ represents a time averaged electric field strength, and Re[K(ω)] represents the real part of the Clausius-Mossotti factor defined by the following formula (3))

$$\begin{matrix}[ Expression 2 ] & \\ \underline{K} ( ω ) = \frac{\underline{{ɛ}_{p}} - \underline{{ɛ}_{s}}}{\underline{{ɛ}_{p}} + \underline{2 {ɛ}_{s}}} & ( 3 )\end{matrix}$$

(where, ∈ₛ and ∈ₚ represent the complex dielectric constants of the solvent and the particle, respectively, defined by the following formula (4))

$$\begin{matrix}[ Expression 3 ] & \\ \underline{ɛ} = ɛ - \frac{σ}{ω} j & ( 4 )\end{matrix}$$

(where, σ represents an electrical conductivity, e represents a dielectric constant, and ω represents an angular frequency defined by 2πf, f representing the frequency of the applied alternating electric field). According to the formula (2), it is shown that the dielectrophoretic force is proportional to the square of an electric field gradient. Accordingly, stronger dielectrophoretic force acts on cells near the area where lines of electric force are concentrated and a high electric field gradient is generated. As a result, large repulsive force can be applied on the cells, making it possible to pattern the cells more sufficiently. In other words, in a region with low electric field strength and with a large dielectrophoretic force, it is possible to more sufficiently arrange the cells, and thereby to pattern the cells more clearly. In the present invention, cells can be patterned by utilizing the phenomenon in which cells present in a region with high electric field strength as described above moves to a region with low electric field strength upon receipt of repulsive force caused by negative dielectrophoresis. In addition, in such a method for cell patterning, a clearer cell pattern can be formed by making the dielectrophoretic force acting on the cells larger, that is, by making the repulsive force acting on the cells larger, within a range where the cells are not damaged.

Such a position with low electric field strength can not be specified, since the position has a relatively weak electric field and is located within a non-uniform electric field; thus the position is determined relatively in accordance with the strength, frequency, and the like of the voltage to be applied. However, in a case where the multiple electrodes generate a plurality of electric fields having electric-field-strength maximum values of 8×10⁴ V/m or more on the cell culture substrate, the position with low electric field strength is preferably the halfway region between the maximum points of the electric fields adjacent to each other that satisfy the following conditions. Specifically, the electric-field-strength maximum value is 8×10⁴ V/n or more (more preferably, 8×10⁴ to 10×10⁴ V/m, further preferably, approximately 9×10⁴ V/m), and the space between the electric-field-strength maximum points is 30 to 200 μm (more preferably 30 to 150 μm). In such a region, arranged cells are more sufficiently urged toward the cell culture substrate, enabling the cells to adhere onto the cell culture substrate more sufficiently. In regions other than such a region, cells tend to be patterned insufficiently or patterned cells, if any, tend to be killed. Such a halfway region between the maximum points of the electric fields is preferably a region within 30 μm (more preferably 20 μm, further preferably 10 μm) from the center of the maximum points.

In the present invention, the magnitude of the dielectrophoretic force can not be specified, since the magnitude varies depending on the kind of cells to be used, the kind of solvent to be used, the design of the apparatus, the magnitude and the frequency of a voltage to be applied, and the like. However, when a voltage of approximately 10 to 14 Vpp (Vpeak-to-peak) is applied, the magnitude of the dielectrophoretic force is preferably 100 pN or more. At a position where such the magnitude of dielectrophoretic force is 100 pN or more and the electric field strength is weak, it becomes possible to pattern cells more sufficiently. As a result, the cells tend to adhere more sufficiently on the cell culture substrate, when the cell culture substrate is detached from the apparatus.

A method also adoptable in the present invention is a method wherein a plurality of cell suspensions are prepared as the cell suspension, the plurality of cell suspensions are introduced one after another into the region, by selecting a position with low electric field strength depending on cells in each suspension, a plurality of cells are arranged onto the cell culture substrate one after another so as to obtain the cell culture substrate on which the plurality of cells are arranged in a predetermined pattern. In such a method, plurality of cells are arranged one after another at their respective predetermined positions on the cell culture substrate by appropriately changing the frequency or the like of the voltage applied depending on the kinds of used cell suspensions, to appropriately control the position with low electric field strength for each kind of cells. In this way, multiple kinds of cells are arranged in a predetermined pattern, which allows a patterned co-culture of the plural kinds of cells.

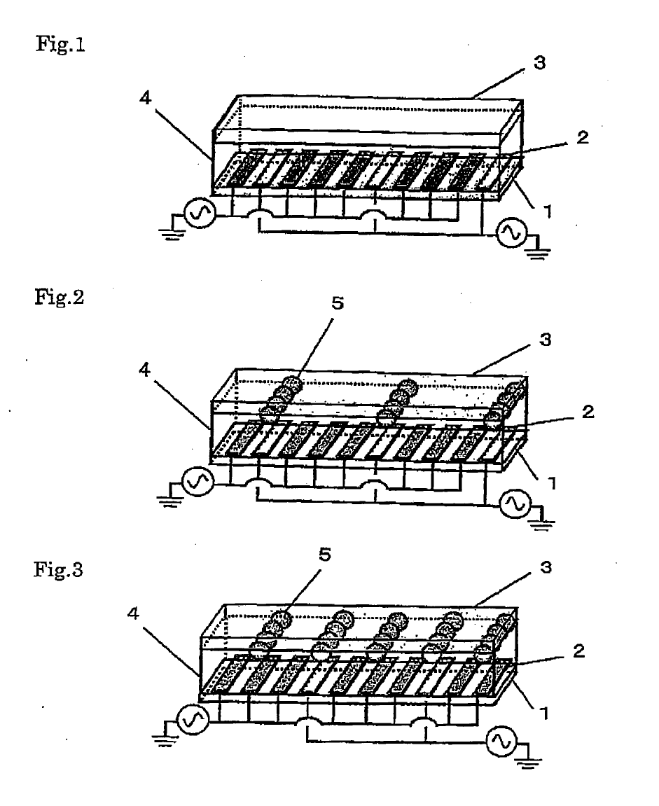

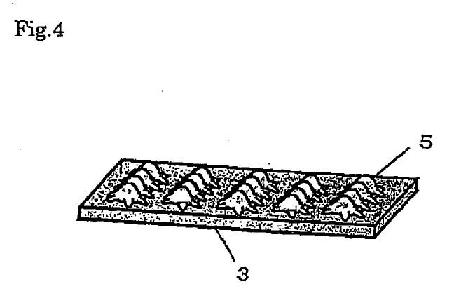

Hereinafter, as a more specific example, description will be made of a method for cell patterning in which the apparatus shown in FIG. 1 is used and an alternating voltage is applied. In the apparatus, an interdigitated array electrode is employed as the electrodes 2. In the interdigitated array electrode, four electrodes operate independently. The phase of the alternating voltage at the micro-band electrodes disposed every fourth electrode is different from that at the other electrodes. First, a cell suspension is introduced into the region between the electrode substrate 1 and the cell culture substrate of the apparatus. Next, the alternating voltage is applied, and cells are subjected to negative dielectrophoresis. Thereby the cells are guided to the position with low electric field strength. In the present embodiment, the position with low electric field strength is regions on the cell culture substrate, and positions each facing one electrode hating a different phase from that of the other electrodes among successively arranged four electrodes. As a result, in the present embodiment, the cells are linearly arranged in each position located on the cell culture substrate and facing one electrode having the different phase (refer to FIG. 2). Thereafter, when the combination of the electrodes to which the alternating voltages is applied is changed in order to change the position with low electric field strength, cells can be guided to regions different from those in a first pattern (refer to FIG. 3). Then, after the cells are arranged in the above described manner, the cell culture substrate is detached. Thus, the cell culture substrate as shown in FIG. 4 can be obtained on which the cells are arranged in the same pattern as the predetermined pattern of the electrodes. Note that, if multiple cell culture media are prepared and changed one after another for use in repeating the cell arrangement one after another, xenogeneic cells can be arranged separately onto the cell culture substrate, which allows a patterned co-culture of the multiple kinds of cells.

### EXAMPLES

Hereinafter, the present invention will be more specifically described on the basis of Examples and Comparative Example; however, the present invention is not limited to Examples below.

### Production Example 1

### Production of Electrode Substrate

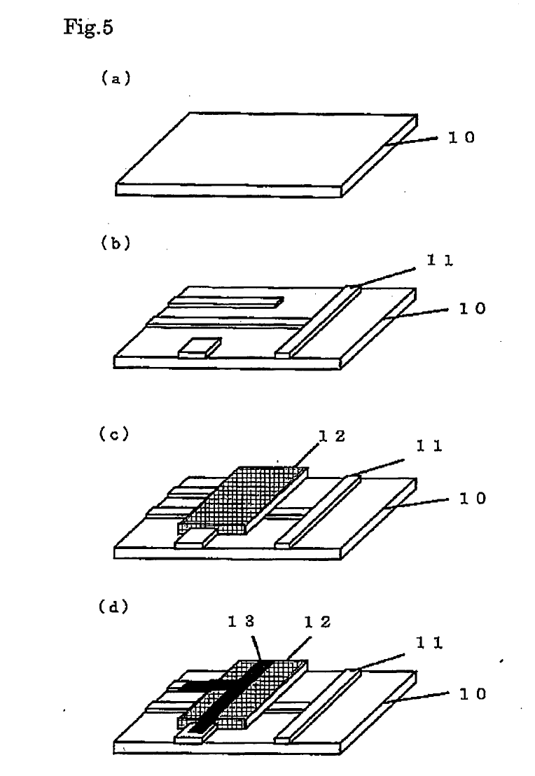

An electrode substrate was produced in which an IDA electrode with four independently-operating electrodes was formed by photolithography. FIG. 5 shows an outline view of the process of producing such an electrode substrate.

First, an ITO electrode substrate 10 (manufactured by Sanyo Vacuum Industries Co., Ltd.: 25 mm×35 mm) as shown in FIG. 5(a) was washed. Then the ITO electrode substrate 10 was spin-coated with hexamethyldisilasane, and a positive photoresist (manufactured by Shipley Company L. L. C under the trade name “S-1818”) in this order. Thereafter, the ITO electrode substrate 10 was baked for 3 minutes under a temperature condition of 110° C., subjected to UV irradiation (500 W, 10 seconds) through a photomask having a predetermined IDA electrode pattern. Then, the ITO electrode substrate 10 was immersed into a liquid developer (manufactured by Shipley Company L. L. C under the trade name “MICROPOSIT MF CD-26” to obtain an IDA electrode pattern (electrode wirings 11) of the photoresist (FIG. 5(b)).

Next, the photoresist was baked for 60 minutes under a temperature condition of 120° C., and part coated with no resist is removed by electrochemical etching. In the electro-chemical etching, a platinum plate was used as the counter electrode. The electro-chemical etching was performed in a 5:4:5HCl/HNO₃/H₂O solution, while applying an alternating voltage (500 Hz, 20 Vpp) for 20 minutes by use of a function generator (manufactured by NF corporation under the trade name “WF1966”). Thereafter, the substrate was subjected to ultrasonic treatment in acetone to remove the resist mask, and then subjected to oxygen plasma treatment for 30 seconds under a condition of 100 W by use of “LTA-101” manufactured by Yanaco Inc. to remove small organic matters. Next, the electrode substrate was spin-coated with a negative photoresist (manufactured by MicroChem. Corp. under the trade name “SU-8 2002”) for 30 seconds under a condition of 3000 rpm, and was subjected to exposure and development to form a bridge 12 that straddles the electrode wirings 11 and that has a predetermined shape (FIG. 5(c)).

Thereafter, the electrode substrate was subjected to oxygen plasma treatment and heat bake (160° C., 30 minutes), then uniformly applied with a photoresist (manufactured by Shipley Company L. L. C under the trade name “S-1818”) again to thereby form such a resist pattern that straddles the bridge 12 and the underlying electrode wirings 11. A gold electrode that straddles the bridge 12 and the underlying electrode wirings 11 was formed by a combination of sputtering deposition of Ti/Au (with “L-332S-FH” manufactured by Canon ANELVA Engineering Corporation) and a lift-off method using acetone (FIG. 5(d)). Then, an exposed part (1.8 mm×0.75 mm) of an electrode 13 is defined by use of a negative photoresist (manufactured by MicroChem. Corp. under the trade name “SU-8 2002”).

In this production example, with the above process, an IDA electrode of four-independently-operating-electrode type was formed.

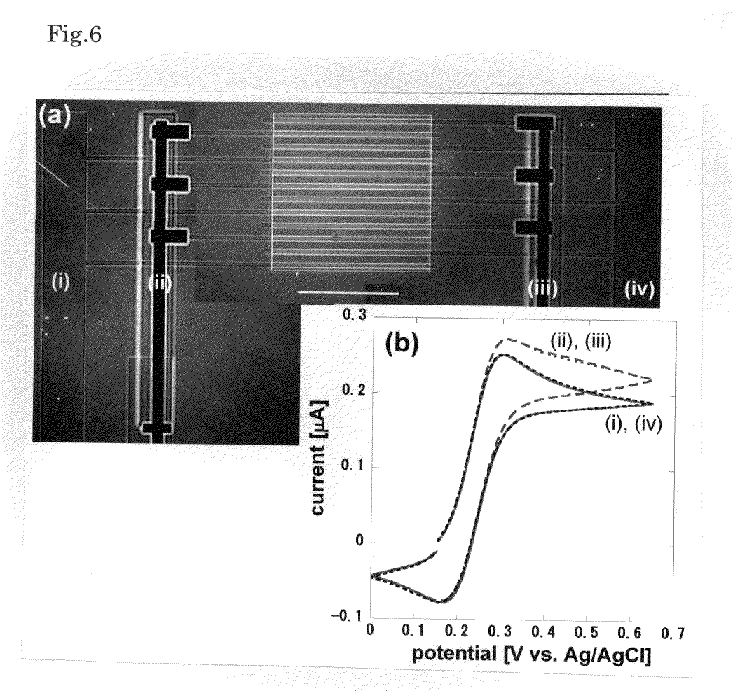

In the electrode, four micro-band electrodes (electrode wirings 11) were taken as a basic unit, the basic unit was repeated three times, and the micro-band electrodes were alternately disposed in a meshed comb shape. The micro-band electrodes were made to have a width of 50 μm and arranged at a 100-μm pitch. In addition, for simplifying wiring arrangement, the electrodes were wired in a way that four contacting pads and each micro-band electrode were connected. As described above, the bridge was formed of a negative resist on part where electrode wirings crossed each other, and the gold electrode that extends on the bridge was formed. Moreover, the area of electrode to be in direct contact with a solvent above the electrode substrate was set to 12×50 μm×0.75 mm, and part other than the region was insulating-coated with a negative resist. FIG. 6(a) shows an optical micrograph of the thus obtained XDA electrode (electrode substrate) of four-independently-operating-electrode type.

As apparent from the optical micrograph shown in FIG. 6(a), it was observed that, in the obtained electrode substrate, 12 micro-band electrodes each having a width of 50 μm were arranged at a 100 μm pitch in a 1.8 mm×0.75 mm square at the center of the obtained electrode substrate and that all the regions other than the central electrode portion were insulating-coated with the negative resist. The especially black parts represent the gold electrodes formed on the bridges, and thus it was observed that each gold electrode and the underlying ITO electrodes were connected. Further, it was revealed that three micro-band electrodes were disposed to one lead region, and 12 micro-band electrodes in total were disposed in the obtained electrode substrate.

Next, the obtained electrode substrate was subjected to electrochemical measurement that was conducted as follows. Specifically, the electro-chemical measurement on the electrode substrate was performed in a 4 mM aqueous solution of K₄[Fe(CN)₆] (manufactured by KANTO CHEMICAL CO., INC.) containing 100 mM of KCl. For the measurement, the obtained electrode substrate was used as a working, a platinum plate was used as a counter, and Ag/AgCl was used as a reference electrode. In the measurement, the electrode substrate was provided with a solution chamber made of acrylic (10×20×5 mm), while a silicone sheet (with a thickness of 2 mm) with a 6×6 mm square hole was interposed between the electrode substrate and the solution chamber. Then, 1 mL of the K₄[Fe(CN)₆] aqueous solution was filled into the solution chamber. Thereafter, cyclic voltammetry was conducted at a scan speed of 20 m V/s by use of a potentiostat (manufactured by HOKUTO DENKO CORPORATION under trade name of “HA1010mM8”) computer-controlled by a software programmed by the present inventors. FIG. 6(b) shows the obtained cyclic voltammogram.

As apparent from the results shown in FIG. 6(b), all electrodes (i) to (iv) shown in the optical micrograph in FIG. 6(a) provided almost the same peak current values, and the shapes of cyclic voltammograms were sigmoidal shapes that are characteristic to CV measurement. In addition, it was observed that the electrodes (electrodes (ii) and (iii)) that were connected through the bridge structures had slightly larger peak currents than the underlying ITO electrodes (electrodes (i) and (iv)). It is speculated that this result was caused because gold of the electrodes on the bridges and gold of the electrodes connecting the underlying ITO electrodes were not completely insulating-coated.

In addition, since three micro-band electrodes connected to one lead region was present at a pitch of 550 μm, and thus was separated enough. Hence, the peak current value per micro-band electrode was calculated, and the peak current value per lead was calculated from the peak current value per micro-band electrode. Note that the peak current value Ip of the micro-band electrodes was obtained by the following formula (5):

$$\begin{matrix}[ Expression 4 ] & \\ {I}_{p} = {nFc}^{*} Db ( 0.439 p + 0.713 {p}^{0.108} + \frac{0.614 p}{1 + 10.9 p} ) \\ p = \sqrt{\frac{{nFw}^{2} v}{RTD}} & ( 5 )\end{matrix}$$

(where, F represents the Faraday constant (=9.648×10⁴ Cmol⁻¹), R represents the gas constant (=8.314 Jmol⁻¹K⁻¹), T represent the absolute temperature (=298 K), D represents the diffusion constant of Fe[(CN)₆]⁴⁻ (=6.5×10⁻¹⁰ m²s⁻¹), c* represents the bulk concentration of Fe[(CN)₆]⁴⁻ (=4 molm⁻³), w represents the width of the electrode (=5.0×10⁻⁵ m), b represents the length of the electrode (=7.5×10⁻⁴ m), and v represents the scan speed (=2×10⁻² Vs⁻¹).)

In this way, the peak current value (theoretical value) of one lead region can be derived as 0.89 μA. The theoretical value is somewhat larger than the actual measurement results (0.25 to 0.28 μA); however, the actual measurement current value was almost the same as the theoretical value. Therefore, it was found out that, in the obtained IDA electrode, four electrodes function as electrodes completely independently and accurately.

### Production Example 2

### Production of Cell Patterning Apparatus

An apparatus with a structure shown in FIG. 1 was produced. In such an apparatus, the electrode substrate (the IDA electrode with four independently-operating electrodes) produced in Production Example 1 was used as an electrode substrate 1. “TL-41MS-06K” manufactured by Lintec Corporation was used as a spacer 4. A culture slide (a polystyrene cell culture slide: 25×25 mm manufactured by Nalge Nunc International K.K.) was used as a cell culture substrate 3. The space between the electrode substrate 1 and the cell culture substrate 3 was set to 30 μm.

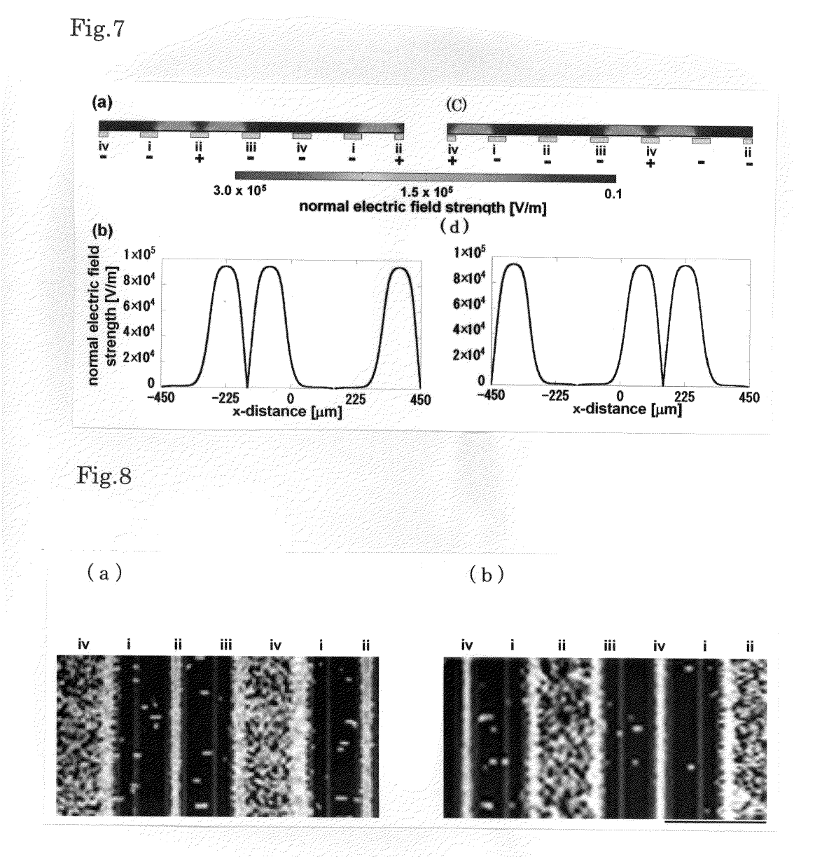

The electric field strength in such a cell patterning apparatus was calculated by use of finite element analysis software “COMSOL Multiphysics 3.1a (manufactured by Comsol, Inc. in Sweden). The calculation was conducted in a three dimensional model. The dimensions of the model was set to length (x axis) 900 μm×width (y axis) 10 μm×height (z axis) 30 μm. The electrode substrate was taken as a base surface (z=0), and the electric field strength in x-z plane at y=0 was calculated, assuming a case where a voltage of +6 V was applied to a positive electrode side (electrode (ii) in FIG. 6(a)), and a voltage of −6 V was applied to a negative electrode side (electrodes (i), (iii), and (iv) in FIG. 6(a)). Note that the device was assumed to be filled with water (∈=78 ∈₀). FIG. 7 shows the result of such an electric field strength analysis.

In FIG. 7(a), bright regions represent regions with high electric field strength, whereas dark regions represent regions with low electric field strength. FIG. 7(b) is a cross-sectional view of the electric field strength on the model of the top surface (z=30 μm). The results shown in FIGS. 7(a) and (b) shows that lines of electric force concentrate on the electrode (ii), that electric field strength is sharply weaken above the electrode (ii), and that regions with low electric field strength extend above the three other electrodes (electrodes (i), (iii) and (iv)).

Next, a suspension containing 2.74% by mass of polystyrene fine particles (with a diameter of 2 μm, manufactured by Polysciences, Inc.) and 1.37% by mass of dimethylsulfoxide as a solvent was introduced into the region between the electrode substrate 1 and the cell culture substrate 3 of the cell patterning apparatus. Then, an alternating voltage of 1 MHz, 20 Vpp was applied to the electrodes, in order to subject the polystyrene fine particles to negative dielectrophoresis. FIG. 8(a) shows the obtained optical micrograph.

As apparent from the results shown in FIG. 8(a), it was observed that the polystyrene fine particles caused to move to regions with low electric field strength by the negative dielectrophoresis. In particular, the polystyrene fine particles located in a region right above the electrode (ii) on the cell culture substrate were arranged within the range that were substantially equivalent to the width of the electrode (ii). It was found out that a clear pattern of the polystyrene fine particles was formed above one electrode (electrode (ii)) by the negative dielectrophoresis. In a region on the substrate right above the three other electrodes, the fine particles were widely distributed, and thereby a clear pattern was not formed. This can be speculated as follows. The fine particles near the region right above the electrode (ii) where a high electrical gradient was locally formed were sufficiently arranged by the dielectrophoretic force, whereas the fine particles in the regions right above the three other electrodes were not sufficiently arranged. In the region right above three other electrodes, the distance between the formed electrical gradients was large, and thus sufficient dielectrophoretic force was not applied to the fine particles, thereby leaving the fine particles suspended. These results show that cells can be arranged in a clearer pattern in a halfway region between maximum points of electric fields being adjacent to each other and satisfying conditions that the electric-field-strength maximum value of each electric field is 8×10⁴ V/m or more, and the space between electric-field-strength maximum points is 30 to 200 μm.

Next, by use of the above-described cell patterning apparatus in which the electrode (iv) was used as the positive electrode and the electrodes (i) to (iii) were used as the negative electrodes, measurement of electric field strength and negative dielectrophoresis with the polystyrene fine particles were conducted. FIGS. 7(c), (d) and FIG. 8(b) show the obtained results. It was found out from the results shown in FIGS. 7(c), (d) and the like that there were no changes in the magnitude of the electric field strength (FIG. 7(d)) and in the patterning precision of the fine particles (FIG. 8(b)), except that the electric field strength was sharply weaken above the electrode (iv) These results revealed that, by controlling application electrodes, it is possible to control the position where the fine particles are arranged, that is, to arrange cells in any pattern by use of the electrode substrate with multiple electrodes.

### Production Example 3

### Culture of Cells (C2C12))

A mouse myoblast cell line (C2C12) was cultured. Specifically, the undifferentiated mouse myoblast cell line (C2C12) was cultured under conditions of 37° C., 5% by volume of CO₂, and water vapor saturation in a Dulbecco's modified Eagle's minimal essential medium (DMEM: manufactured by Gibco) added with lot by volume of immobilized FBS (manufactured by Gibco), 25 U/mL of penicillin, and 25 μg/mL of streptomycin (manufactured by Gibco).

### Production Example 4

### Culture of Cells (3T3 Swiss-Albino

Mouse fibroblast cells (3T3 swiss-albino) were cultured. Specifically, the mouse fibroblast cells (3T3 swiss-albino) were cultured in a RPMI (manufactured by Gibco) 1640 medium added with lot by volume of immobilized FBS (manufactured by Gibco), 50 U/mL of penicillin, and 50 μg/mL of streptomycin (manufactured by Gibco).

### Production Example 5

### Production of Cell Suspension

A cell suspension was prepared by use of confluent cultured C2C12 cells. Specifically, the cell suspension was prepared as follows. The cultured C2C12 cells were treated with an EDTA solution containing 0.25 w/wt of trypsin so as to be floated, and then centrifugated at 1500 rpm for 3 minutes. Then, the cells were resuspended in a DMEM medium (a differentiation medium) containing 2% by volume of horse serum, 25 U/mL of penicillin, and 25 μg/mL of streptomycin to obtain a cell concentration of 2.0×10⁷ cells/mL. The cell suspension was stored under a temperature condition of 4° C., until the cell suspension was used.

### Test Example 1

Cell suspensions were prepared in which the 3T3 fibroblast cells (Production Example 4) were suspended in the RPMI medium added with a 250 mM sucrose aqueous solution while the electrical conductivities were adjusted to various values. Then, each cell suspension was introduced into the region between the electrode substrate and the cell culture substrate of the cell patterning apparatus. Thereafter, the cells were subjected to dielectrophoresis (with a voltage of 9.5 Vpp) Note that a case where the cells were attracted to the edge of the IDA electrode is defined as exertion of positive dielectrophoresis, whereas a case where the cells were patterned linearly on the cell culture substrate is defined as exertion of negative dielectrophoresis.

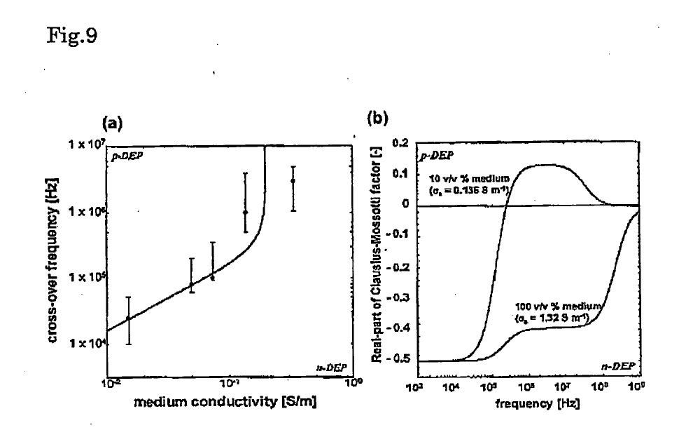

When the cells were suspended in a solvent with a low electrical conductivity (σₛ=0.015 Sm⁻¹), negative dielectrophoresis started to occur at approximately 10 kHz. Thereafter, the force of the negative dielectrophoresis was weakened, as the frequency was increased. At a frequency of approximately 25 kHz, dielectrophoretic force ceased to act on the cells. When the frequency was further increased, positive dielectrophoresis started to occur. FIG. 9(a) shows a graph illustrating a relationship between the electrical conductivity of the medium and the frequency (cross-over frequency) at which dielectrophoretic force ceased to act. The solid line in the graph is a line obtained by fitting the theoretical cross-over frequency of the cells to the experimental values, under the assumption that the protoplast model of a conductive sphere coated with an insulating thin film holds true for the cells.

As apparent from the graph shown in FIG. 9(a), it was found out that, as the electrical conductivity of the solvent increased, the cross-over frequency was shifted to the high frequency side. It was also found out that, in 25 v/v % medium (σₛ=0.33 Sm⁻³), negative dielectrophoresis occurred up to approximately 3 MHz. Moreover, it was found out that, when the medium concentration was increased to 50, 75, and 100 v/v %, the negative dielectrophoresis acted over a wide range of 100 kHz to 10 MHz. Note that the Clausius-Mossotti factor in the protoplast model is represented by the following formula (6):

$$\begin{matrix}[ Expression 5 ] & \\ \underline{K} ( ω ) = - \frac{{ω}^{2} ( {τ}_{s} {τ}_{m} - {τ}_{c} {τ}_{m}^{′} ) + j ω ( {τ}_{m}^{′} - {τ}_{u} - {τ}_{m} ) - 1}{{ω}^{2} ( {τ}_{c} {τ}_{m}^{′} + 2 {τ}_{s} {τ}_{m} ) - jω ( {τ}_{m}^{′} + 2 {τ}_{s} + {τ}_{m} ) - 2} & ( 6 )\end{matrix}$$

(where, τₘ=cₘr/σ_{c}, τ_{c}=∈_{c}/σ_{c}, τₛ=∈ₛ/σₛ, τ′ₘ=cₘr/σₛ, cₘ represents the cell membrane capacitance [Fm⁻²], and the subscripts c and s represent a cytoplasm and a solvent, respectively). The frequency at which Re[K]=0 was calculated by use of a cell radius r=6.7×10⁻⁶ [m], and the dielectric constant of the solvent ∈ₛ=78 ∈₀ as physical values. For the calculation, Mathematica 5.1 (manufactured by WOLFRAMRESEARCH, Inc.) was used, and the least square method was used for fitting. With such fitting, the following physical values of the cell were identified: the electrical conductivity of cytoplasm σ_{c}=0.198 Sm⁻¹; the dielectric constant of the cytoplasm ∈_{c}=60 to 78 ∈₀; and the cell membrane capacitance Cₘ=0.02 Fm⁻². With these physical values, Re[K] was plotted against change in frequency by use of formula (6). FIG. 9(b) shows the obtained result.

As apparent from the result shown in FIG. 9(b), it was found out that, in a solvent with a low electrical conductivity, negative dielectrophoresis occurs in the low frequency side and in a region of several tens to several hundreds of MHz, while positive dielectrophoresis occurs in a region between the low frequency side and the region of several tens to several hundreds of MHz. On the other hand, it was found out that, in the medium with a high electrical conductivity, negative dielectrophoresis occurs in almost all frequency regions. Meanwhile, it was observed that, in a solvent with a high electrical conductivity, bubbles were formed and the electrodes were corroded due to an electrode reaction, when the frequency is several hundreds of kHz or less. These results revealed that the frequency of the voltage is preferably controlled to approximately 1 MHz in a case where cells are patterned by use of the above-described apparatus.

### Example 1

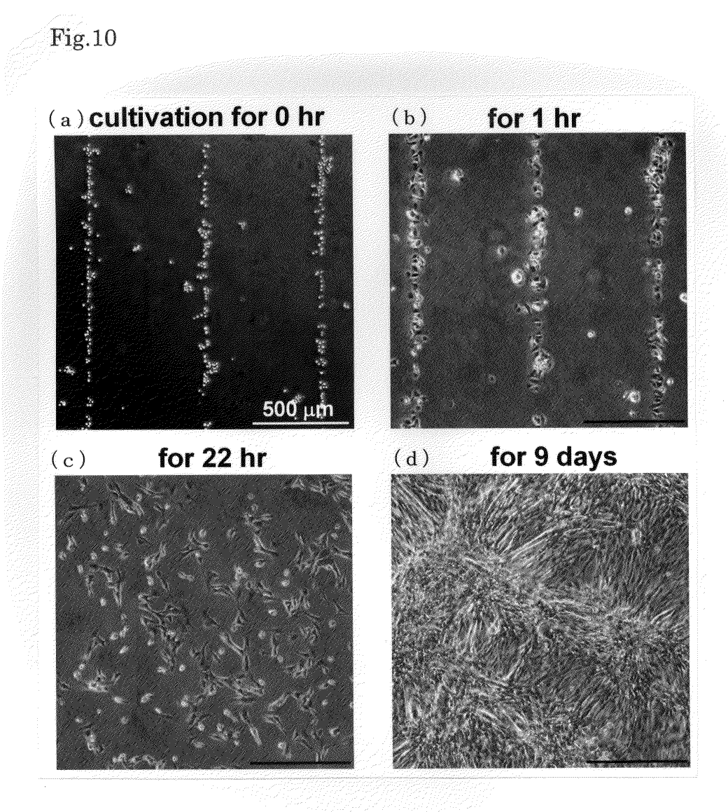

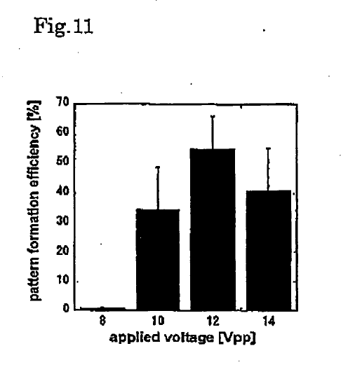

The C2C12 myoblast cell suspension (Production Example 5) suspended in the differentiation medium was introduced into the region between the electrode substrate 1 and the cell culture substrate 3 of the cell patterning apparatus (Production Example 2), and subjected to application of an alternating voltage (12 Vpp). In such a cell patterning apparatus, the electrode (ii) in FIG. 6(a) was used as the positive electrode, and the electrodes (i), (iii) and (iv) in FIG. 6(a) were used as the negative electrodes. Then, an alternating voltage (1 MHz, 12 Vpp) was applied for 5 minutes to arrange cells on the cell culture substrate disposed above the electrodes. FIG. 10 shows an optical micrograph of the thus obtained cell culture substrate detached from the apparatus.

As apparent from the optical micrograph shown in FIG. 10(a), it was observed that cells which nonspecifically adhered onto the cell culture substrate in a region above the electrode (i), (iii), or (iv) and cells which were located and were not patterned in the region above the electrodes (i), (iii), and (iv) where the dielectrophoresis was week were removed along with the separation of the cell culture substrate from the device. It was also observed that only cells arranged linearly in the region above the electrode (ii) were sufficiently arranged onto the cell culture substrate, and that the cells were patterned.

Next, the obtained cell culture substrate was immersed into a medium, and the cells are cultured. FIGS. 10(b) to (d) show optical micrographs of the cell culture substrate after lapse of one hour, 22 hours, and 9 days, respectively.

As apparent from the results shown in FIGS. 10(b) to (d), the cells after one-hour culture changed their shapes from spherical shapes to flattened shapes. This shows that the cells bonded to the substrate. After 22-hour culture, no patterned structures were observed any more. On the ninths of the culture, distinct tubular structures were observed. These results shows that myoblast cells exposed to an electric field do not lose their differentiation potency and can form myotubes.

According to these results, it was revealed that the present invention can achieve rapid formation of a cell pattern by utilizing negative dielectrophoresis. In addition, it was revealed that the present invention can control cell positioning without performing surface treatment on a cell culture substrate, making it possible to easily trace the morphological change, the mobility, and the growth of cells with lapse of culture time.

### Example 2

Cells were arranged onto cell culture substrates as similar to Example 1, except that various magnitudes of voltage were applied. FIG. 11 shows a graph of pattern efficiency (eₚ) against the various voltages.

As apparent from the results shown in FIG. 11, formation of a cell pattern by voltage application was observed when a voltage of 8 Vpp was applied; however, the pattern efficiency was not necessarily sufficient after detachment of the cell culture substrate from the apparatus. On the other hand, it was observed that, as the voltage applied was increased, the pattern efficiency increased. The pattern efficiency reached the maximum value thereof at application of 12 Vpp. It was also observed that, when a greater voltage of 14 Vpp was applied, the pattern efficiency decreased. It is speculated that this decrease occurred because of the following reasons. Specifically, since part of the cells were damaged by the application of this high voltage, the cells once adhered to the substrate were not firmly bonded onto the substrate, thereby leading to separation of the cells from the substrate in a one-hour culture.

### Example 3

The cell patterning apparatus obtained in Production Example 2 were used herein, and cell suspensions were introduced thereinto one after another. By controlling the region with low electric field strength for each cell suspension, cells were sequentially arranged and thus patterned in different regions.

First, a cell suspension (Production Example 5) containing the C2C12 myoblast cells suspended in the differentiation medium was introduced into the region between the electrode substrate 1 and the cell culture substrate 3 of the cell patterning apparatus. While using the electrode (ii) shown in FIG. 6(a) as the positive electrode and the electrodes (i), (iii) and (iv) in FIG. 6(a) as the negative electrodes, an alternating voltage (12 Vpp, 1 MHz) was applied between the electrodes for 5 minutes to perform a first patterning on the regions on the cell culture substrate, the regions being above the electrode (ii).

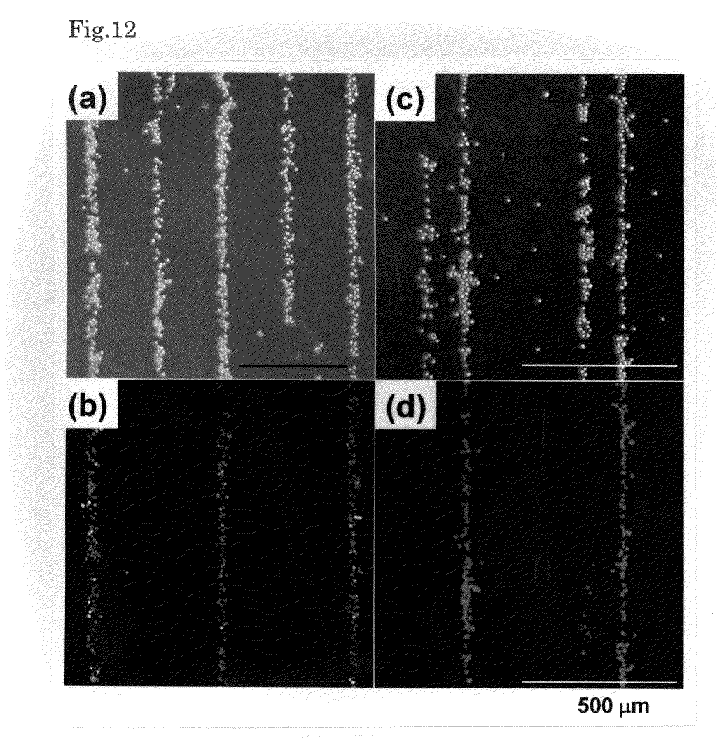

Next, a fluorescent dye CMFDA was introduced into the region to stain the patterned cells. Then, the region was replaced with minimal serum free medium Opti Mem (manufactured by Gibco) containing 10 μM of CMFDA. The cells were incubated at room temperature for 20 minutes. Subsequently, the inside of the device was washed with a differentiation medium. Thereafter, another cell suspension (Production Example 5) was introduced into the region. Then, while using the electrode (i) as the positive electrode and the electrodes (ii), (iii) and (iv) as the negative electrodes, an alternating voltage (12 Vpp, 1 MHz) was applied between the electrodes for 5 minutes to perform a second patterning. FIGS. 12(a) and (b) show optical micrographs of the obtained cell culture substrate. Note that FIG. 12(b) is a photograph at a time when the cells were caused to emit fluorescent.

As apparent from the results shown in FIGS. 12(a) and (b), it was observed that the cells stained with the fluorescent dye and the cells without stain were alternately patterned at a 250 μm pitch.

### Example 4

Cells were patterned on multiple regions as similar to Example 3, except that the electrode (iv) was used as the positive electrode and the electrodes (i), (ii) and (iii) were used as the negative electrode in the second patterning. FIGS. 12(c) and (d) show optical micrographs of the obtained cell culture substrate. Note that FIG. 12(d) is a photograph at a time when the cells were caused to emit fluorescent. As apparent from the results shown in FIGS. 12(c) and (d), it was observed that the cells without stain were arranged in regions each located on the left of the cells stained with the fluorescent dye, while spaced therefrom by 100 μm.

From the results shown in Examples 3 and 4, it was found out that, it is possible to arrange multiple cells onto the cell culture substrate in a predetermined pattern by introducing plural cell suspensions into the region one after another, and by controlling the position with low electric field strength depending on each cell thus introduced. In addition, it was found out that the present invention makes it possible to easily pattern different kinds of cells indifferent regions, respectively, without requiring pre-treatment on the cell culture substrate.

### INDUSTRIAL APPLICABILITY

As described above, the present invention can provide a method for cell patterning which: eliminates the need for forming, in advance, a micrometer-order pattern on a cell culture substrate to arrange cells; allows cells to be efficiently arranged onto the cell culture substrate in a predetermined pattern; and enables an electrode substrate to be used repeatedly by detaching the electrode substrate from the cell culture substrate.

Therefore, the method for cell patterning of the present invention is especially useful as a technique for performing in vitro reconstitution of an in vivo cellular environment. Accordingly, the present invention can be applied to various fields such as drug screening, and elucidation of intercellular communication and cell-extracellular matrix communication for regenerative medicine.

1. A method for cell patterning, using an electrode substrate including a plurality of electrodes and a cell culture substrate disposed so as to face the electrode substrate, the method comprising the steps of:
introducing a cell suspension containing cells into a region between the electrode substrate and the cell culture substrate;
applying a voltage to the electrodes to generate a non-uniform electric field in the region; and
arranging the cells at a position with low electric field strength on the cell culture substrate by utilizing negative dielectrophoresis so as to obtain the cell culture substrate on which the cells are arranged in a predetermined pattern.

2. The method for cell patterning according to claim 1, wherein
a plurality of cell suspensions are prepared as the cell suspension,
the plurality of cell suspensions are introduced one after another into the region, and
by selecting a position with low electric field strength depending on the cells in each dell suspension, a plurality of cells are arranged onto the cell culture substrate one after another so as to obtain the cell culture substrate on which the plurality of cells are arranged in a predetermined pattern.

3. The method for cell patterning according to claim 1, wherein
in a case where the plurality of electrodes generate a plurality of electric fields having electric-field-strength maximum values of 8×10⁴ V/m or more on the cell culture substrate, the position with low electric field strength is a halfway region between maximum points of the electric fields being adjacent to each other and satisfying conditions that an electric-field-strength maximum value of each electric field is 8×10⁴ V/m or more, and that a space between the electric-field-strength maximum points is 30 to 200 μm.

4. The method for cell patterning according to claim 1, wherein
in a case where the plurality of electrodes generate a plurality of electric fields having electric-field-strength maximum values of 8×10⁴ V/m or more on the cell culture substrate, the position with low electric field strength is a halfway region between maximum points of the electric fields being adjacent to each other and satisfying conditions that a electric-field-strength maximum value of each electric field is in a range of 8×10⁴ to 10×10⁴ V/m, and a space between the electric-field-strength maximum points is 30 to 150 μm.

5. The method for cell patterning according to claim 1, wherein
a distance between the electrode substrate and the cell culture substrate is 30 to 50 μm.

6. The method for cell patterning according to claim 1, wherein
a content of the cell in the cell suspension is 5×10⁷ cells/ml or less.

7. The method for cell patterning according to claim 1, wherein
a solvent for the cell suspension has a polarizability larger than a polarizability of the cells.
